(19) United States
(12) Patent Application Publication
(10) Pub. No.: US 2024/0262888 A1
(43) Pub. Date: Aug. 8, 2024

(54) METHOD FOR PREPARING NON-DENATURED TYPE II COLLAGEN

(71) Applicants: Jiaxing Hengjie Biopharmaceutical Co., Ltd., Jiaxing (CN); Fujian Hengjie Bioloical Technology Co., Ltd., Nanping (CN)

(72) Inventors: Meihong Zhu, Zhejiang (CN); Cheng Luo, Jiaxing (CN); Huogen Wang, Jiaxing (CN)

(21) Appl. No.: 17/790,192

(22) PCT Filed: Jul. 29, 2021
(86) PCT No.: PCT/CN2021/109251

§ 371 Date: Jun. 30, 2022

(30) Foreign Application Priority Data
Jul. 19, 2021 (CN) 2021108121930

(51) Int. Cl. C07K 14/78; C12N 9/64; C12P 21/06

(52) U.S. Cl. CPC C07K 14/78; C12N 9/6481; C12P 21/06

(57) ABSTRACT

Embodiments provide a method for preparing non-denatured type II collagen and a product thereof. In the embodiments, chicken breast cartilage is adopted as raw material, through the steps of selecting materials, cleaning, pulverizing, hydrolyzing by enzyme, drying, pulverizing, sieving, etc., the product with a water absorption ratio 10-13 times, a volume expansion ratio 5-7 times, a natural long-chain structure of non-denatured type II collagen retained, and the protein having a content not less than 15% is obtained. Under the premise of improving the content, water absorption and volume expansion coefficient of the non-denatured type II collagen, the product containing the non-denatured type II collagen prepared by the present disclosure has the advantages of fast extraction process and good suspension performance of the product solution, and has good market application prospects.

### TECHNICAL FIELD

The present disclosure provides a method for extracting collagen from animal cartilage, relates to the field of food biotechnology, and in particular, to a method for preparing non-denatured type II collagen.

### BACKGROUND

Collagen is a structural protein widely distributed in mammals. It is an important component of tissues such as skin, bones and joints, and is also one of the main components of the extracellular matrix of multicellular animals. At present, more than 20 types of collagen are known, which can be divided into three categories according to tissue classification: interstitial collagen, basement membrane collagen and cartilage collagen. Among them, type II collagen is mainly distributed in articular cartilage, and a small amount exists in the vitreous body, embryonic cornea and other parts.

Type II collagen is composed of three entangled polypeptide chains, and because it possesses a stable triple helix structure, it mainly plays the role of supporting organs and protecting the body in vivo. Studies in recent years have shown that type II collagen is widely concerned by consumers because of its low immunogenicity and good biocompatibility as a structural and functional nutrient for joints [1].

The currently commercially available type II collagen products are mostly completely enzymatically hydrolyzed collagen products. Due to the limitations of the extraction process and preparation process, under the action of enzymes, high temperature and other factors, type II collagen loses its original triple helix structure, and collagen is decomposed into polypeptides and amino acids below 20,000 Daltons, losing its original preventive and adjuvant treatment functions for rheumatoid arthritis and arthritis^{[2]}.

At present, the processes for preparing a variety of non-denatured type II collagens have been disclosed at home and abroad, such as a method for preparing the cartilage extract containing non-denatured type II collagen disclosed by CN106916870A, including steps of defatting, disinfecting, homogenizing, hydrolyzing by enzyme, filtering, drying and the like. Among them, in the step of hydrolyzing by enzyme: adjusting the pH of the slurry obtained in the step of homogenizing to 2.5˜8.5, adding 0.001%-2% of the enzyme by weight of the cartilage, adding papaya and/or pineapple with 1/20- 1/500 by weight of the cartilage, and then filtering the obtained clear liquid after low-temperature juice extraction; and hydrolyzing by enzyme for 12-48 hours (by Jie Li, Dakai Zhu and Hui Gong. The method for producing non-denatured type II collagen); using pepsin to remove impurities from cartilage for 24 hours, and then hydrolyzing by enzyme and extracting for 24 hours to obtain soluble non-denatured type II collagen protein. U.S. Pat. No. 7,083,820 discloses a method for producing a biologically active product. The chicken breast bone is cleaned, sterilized, minced, mixed (inorganic salt), dried, etc., wherein the above-mentioned steps are operated below 110° F. to obtain type II collagen without altering the original structure of the bioactive product.

### SUMMARY

### Technical Problems

But the disclosed technical method and the prepared non-denatured type II collagen still have problems: first, the product has poor suspension performance in solution, and is easy to stratify; second, for the product preparation and extraction, after that protease or enzyme extraction raw materials are fully hydrolyzed for a long time, the content of non-denatured II collagen is low, and the extraction time is long, which is not conducive to industrial production and promotion.

### Technical Solutions

The present disclosure provides a method for preparing non-denatured type II collagen, aiming at solving one or more problems in the above-mentioned problems.

Concrete technical scheme is as follows:

The present disclosure provides a method for preparing non-denatured type II collagen, characterized by including steps successively of:
(1) selecting raw materials, where the raw materials to be selected are fresh chicken breast cartilage; cutting a part within 3 cm from a tip of the chicken breast cartilage for use after removing fat and muscle manually;
(2) cleaning: cleaning a spare cartilage in step (1) by using clean water with a temperature not higher than 37° C.;
(3) pulverizing: pulverizing the cartilage into cartilage particles below 2 mm by using a pulverizer, after draining the cartilage cleaned in step (2);
(4) hydrolyzing by enzyme: adding water to the cartilage particles obtained in step (3), with an amount of water added 0.5 to 3 times a mass of the cartilage particles (m/V); using acid solution or alkaline solution to adjust a pH value of feed solution between 2.0 and 4.0; adding pepsin by weight of 1.0-5.0% cartilage particles, with an enzymatic hydrolysis temperature not exceed 37° C.; stirring and hydrolyzing by enzyme for 0.5-2.0 h; after the enzymatic hydrolysis, lye is adopted to adjust pH value to 8.5˜9.5, after standing for 15˜60 min;
(5) filtering: filtering enzymatic hydrolyzed solution in step (4) by using a 20-60 mesh sieve, and collecting materials on the sieve;
(6) drying: vacuum-drying or freeze-drying the materials on the sieve, and obtaining a dried product after drying;
(7) pulverizing: pulverizing the dried product, sieving through a 60-200-mesh sieve, and taking the materials under the sieve to obtain non-denatured type II collagen powder.

In the above solution of the present disclosure, firstly by selecting raw materials, selecting chicken breast cartilage, and selecting within 3 cm from the tip of the bone, combined with the improvement of the process, especially the improvement of the enzymatic hydrolysis step, it is possible to obtain non-denatured type II collagen with a content greater than 15%. And the extraction process is quick and easy: the total extraction time does not exceed 4 h.

In an embodiment, the raw materials are selected from a part within 1.5 cm from the tip of the chicken breast cartilage for use.

In an embodiment, the pepsin is added through a load carrier, and the load carrier is food grade silicon dioxide. In this scheme, pepsin is added in the form of a load carrier, such that the degree of enzymatic hydrolysis can be effectively controlled. Combined with the concentration of pepsin and the enzymatic hydrolysis time, the obtained non-denatured II collagen has a high content.

In an embodiment, an addition ratio of the pepsin and the load carrier is (0.3˜0.6):15.

In an embodiment, in step (4), after adding water to the cartilage particles, beating is performed first, and then acid solution or alkaline solution is adopted to adjust the pH value of feed solution between 2.0 and 4.0.

In an embodiment, a beating temperature is controlled below 25° C.

In an embodiment, cleaning times in step (2) are 1 to 3 times, with 10 to 15 minutes each time.

In an embodiment, the enzymatic hydrolysis temperature is 30° C.˜32° C.

A non-denatured type II collagen can be obtained by the method for extracting according to the present disclosure.

### Advantageous Benefit

Compared with prior art, the advantage of the present disclosure is:
(1) The extraction process is fast and simple: the total extraction time does not exceed 4 hours, and the preparation time is significantly shortened compared with the long-time extraction of more than 24 hours in the prior art;
(2) The non-denatured type II collagen content is high, and the product performance is good: in the non-denatured type II collagen prepared by the method of the present disclosure, the non-denatured type II collagen content is greater than 15%, and the water absorption ratio in the aqueous solution is 10-12 times, the volume expansion rate is 5˜7 times;
(3) It has good suspension performance in aqueous solution and is not easy to delaminate. In addition to hard capsules and tablets, it is also suitable for soft capsules, solid beverages, gel candies and other dosage forms.

### BRIEF DESCRIPTION OF DRAWINGS

FIG. 1 is the natural collagen characteristic, regularity striated tissue picture of the non-denatured type II collagen extracted from animal cartilage according to the present disclosure, under transmission electron microscope.

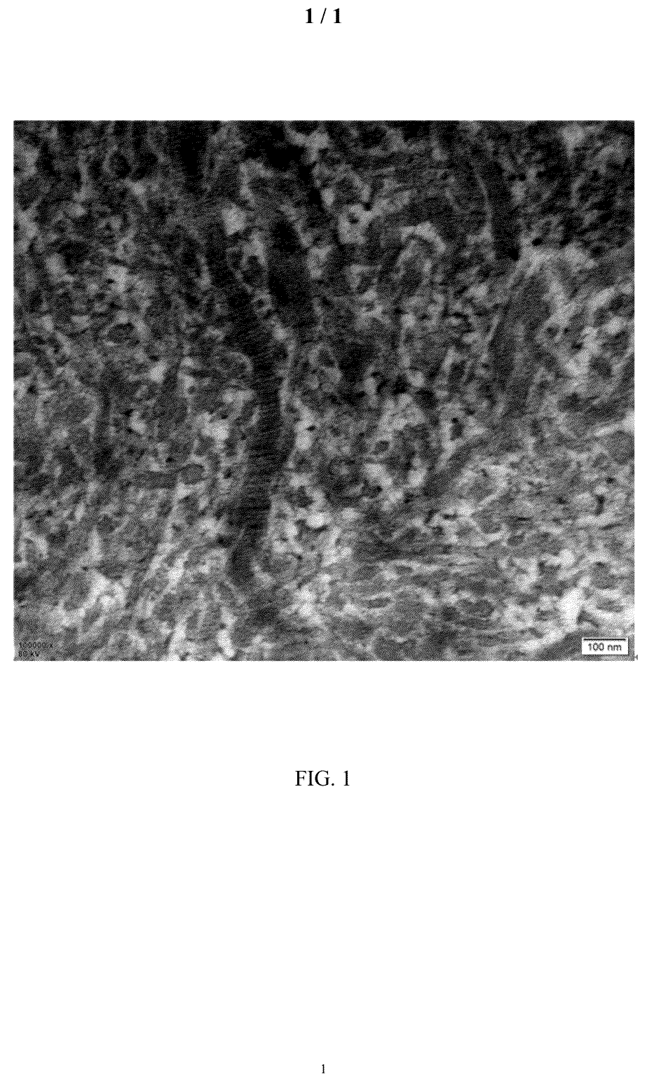

### DESCRIPTION OF EMBODIMENTS

The substantive content of the present disclosure is further described below in conjunction with the examples, but the protection scope of the present disclosure is not limited by this. Although the present disclosure has been described in detail with reference to the preferred embodiments, those skilled in the art should understand that the technical solutions of the present disclosure may be modified or equivalently replaced without departing from the spirit and scope of the technical solutions of the present disclosure.

### Example 1 (Ex. 1)

(1) selecting raw materials, where the raw materials to be selected are fresh chicken breast cartilage; cutting 1.0 kg of cartilage within 1 cm from the tip after removing fat and muscle manually;
(2) cleaning: cleaning by using clean water with a temperature not higher than 37° C. for 1 to 3 times, with 10 minutes each time; then draining;
(3) pulverizing: pulverizing the cartilage into cartilage particles below 2 mm by using a pulverizer, after draining the cartilage cleaned in step (2);
(4) hydrolyzing by enzyme: placing the cartilage particles obtained in step (3) into the reaction tank, adding 1 L of clean water, with the temperature controlled at 35° C.; using acid solution or alkaline solution to adjust a pH value of feed solution between 3.0 and 4.0; adding 20 g of pepsin, stirring and hydrolyzing by enzyme for 2.0 h; after the enzymatic hydrolysis, the pH value being adjusted to 9.0 with lye, and standing for 30 min;
(5) filtering: filtering enzymatic hydrolyzed solution in step (4) by using a 40 mesh sieve, and collecting materials on the sieve;
(6) drying: freeze-drying the materials on the sieve, and obtaining a dried product S1 after drying, with a total of 91.6 g;
(7) pulverizing: pulverizing the dried product, sieving through a 100-mesh sieve, and taking the materials under the sieve.

“Ministry of Health Medicine Standard Biochemical Medicines” (1st volume hexose amino acid method for detecting glycosaminoglycan, 1989) is adopted, glycosaminoglycan content is 22.3% (in chondroitin sulfate, coefficient is calculated as 2.82). The method of GB 5009.5 is adopted, the total protein content is 79.1% (the protein conversion coefficient is calculated as 5.79). The method of GB/T 9695.23 is adopted, the hydroxyproline content is 6.8%. The ELISA kit is adopted for detecting, the non-denatured type II collagen content is 21.88%.

### Water Absorption Multiple Detection:

The above-mentioned materials 20 g under the sieve are taken by weighing in the measuring cylinder with scale, purified water is added to sample all wet and be gelatinous. Product volume is rapidly expanded to 260 mL from 42 mL, and volume expansion is 6.2 times. Quantitative filter paper is used to remove free water. After wetting, the mass of the product is 276.1 g and the water absorption is 256.1 g, which is 12.8 times the weight of the product itself.

### Suspension Ability Test:

The above-mentioned materials 20 g under the sieve are taken, mixed with 5% (w/v) liquid with purified water, left standstill 2 h, without layering.

### Example 2

(1) selecting raw materials, where the raw materials to be selected are fresh chicken breast cartilage; cutting 1.0 kg of cartilage within 2-3 cm from the tip after removing fat and muscle manually;
(2) cleaning: cleaning by using clean water with a temperature not higher than 37° C. for 1 to 3 times, with 10 minutes each time; then draining;
(3) pulverizing: pulverizing the cartilage into cartilage particles below 2 mm by using a pulverizer, after draining the cartilage cleaned in step (2);
(4) hydrolyzing by enzyme: placing the cartilage particles obtained in step (3) into the reaction tank, adding 1 L of clean water, with the temperature controlled at 35° C.; using acid solution or alkaline solution to adjust a pH value of feed solution between 3.0 and 4.0; adding 30 g of pepsin, stirring and hydrolyzing by enzyme for 2.0 h; after the enzymatic hydrolysis, the pH value being adjusted to 9.0 with lye, and standing for 30 min;
(5) filtering: filtering enzymatic hydrolyzed solution in step (4) by using a 40 mesh sieve, and collecting materials on the sieve;
(6) drying: freeze-drying the materials on the sieve, and obtaining a dried product S2 after drying, with a total of 81.5 g;
(7) pulverizing: pulverizing the dried product, sieving through a 100-mesh sieve, and taking the materials under the sieve.

“Ministry of Health Medicine Standard Biochemical Medicines” (1st volume hexose amino acid method for detecting glycosaminoglycan, 1989) is adopted, glycosaminoglycan content is 28.9% (in chondroitin sulfate, coefficient is calculated as 2.82). The method of GB 5009.5 is adopted, the total protein content is 70.3% (the protein conversion coefficient is calculated as 5.79). The method of GB/T 9695.23 is adopted, the hydroxyproline content is 5.2%. The ELISA kit is adopted for detecting, the non-denatured type II collagen content is 16.77%.

### Water Absorption Multiple Detection:

The above-mentioned materials 20 g under the sieve are taken by weighing in the measuring cylinder with scale, purified water is added to sample all wet and be gelatinous. Product volume is rapidly expanded to 200 mL from 39 mL, and volume expansion is 5.1 times. Quantitative filter paper is used to remove free water. After wetting, the mass of the product is 252.2 g and the water absorption is 232.2 g, which is 11.6 times the weight of the product itself.

### Suspension Ability Test:

The above-mentioned materials 20 g under the sieve are taken, mixed with 5% (w/v) liquid with purified water, left standstill 2 h, without layering.

### Example 3

(1) selecting raw materials, where the raw materials to be selected are fresh chicken breast cartilage; cutting 1.0 kg of cartilage within 3 cm from the tip after removing fat and muscle manually;
(2) cleaning: cleaning by using clean water with a temperature not higher than 37° C. for 1 to 3 times, with 10 minutes each time; then draining;
(3) pulverizing: pulverizing the cartilage into cartilage particles below 2 mm by using a pulverizer, after draining the cartilage cleaned in step (2);
(4) hydrolyzing by enzyme: placing the cartilage particles obtained in step (3) into the reaction tank, adding 1 L of clean water, with the temperature controlled at 35° C.; using acid solution or alkaline solution to adjust a pH value of feed solution between 3.0 and 4.0; adding 50 g of pepsin, stirring and hydrolyzing by enzyme for 2.0 h; after the enzymatic hydrolysis, the pH value being adjusted to 9.0 with lye, and standing for 30 min;
(5) filtering: filtering enzymatic hydrolyzed solution in step (4) by using a 40 mesh sieve, and collecting materials on the sieve;
(6) drying: freeze-drying the materials on the sieve, and obtaining a dried product S3 after drying, with a total of 90.1 g;
(7) pulverizing: pulverizing the dried product, sieving through a 100-mesh sieve, and taking the materials under the sieve.

“Ministry of Health Medicine Standard Biochemical Medicines” (1st volume hexose amino acid method for detecting glycosaminoglycan, 1989) is adopted, glycosaminoglycan content is 25.2% (in chondroitin sulfate, coefficient is calculated as 2.82). The method of GB 5009.5 is adopted, the total protein content is 74.5% (the protein conversion coefficient is calculated as 5.79). The method of GB/T 9695.23 is adopted, the hydroxyproline content is 5.9%. The ELISA kit is adopted for detecting, the non-denatured type II collagen content is 18.81%.

### Water Absorption Multiple Detection:

The above-mentioned materials 20 g under the sieve are taken by weighing in the measuring cylinder with scale, purified water is added to sample all wet and be gelatinous. Product volume is rapidly expanded to 212 mL from 40 mL, and volume expansion is 5.3 times. Quantitative filter paper is used to remove free water. After wetting, the mass of the product is 266.1 g and the water absorption is 246.1 g, which is 12.3 times the weight of the product itself.

### Suspension Ability Test:

The above-mentioned materials 20 g under the sieve are taken, mixed with 5% (w/v) liquid with purified water, left standstill 2 h, without layering

### Example 4

(1) selecting raw materials, where the raw materials to be selected are fresh chicken breast cartilage; cutting 1.0 kg of cartilage within 3 cm from the tip after removing fat and muscle manually;
(2) cleaning: cleaning by using clean water with a temperature not higher than 37° C. for 1 to 3 times, with 10 minutes each time; then draining;
(3) pulverizing: pulverizing the cartilage into cartilage particles below 2 mm by using a pulverizer, after draining the cartilage cleaned in step (2);
(4) hydrolyzing by enzyme: adding 1 L of clean water to the cartilage particles obtained in step (3), making a slurry below 25° C.; placing the slurry into the reaction tank, with the temperature controlled at 35° C.; using acid solution or alkaline solution to adjust a pH value of feed solution between 3.0 and 4.0; adding pepsin-loaded food-grade silica including 10 g of pepsin and 500 g of food-grade silica, stirring and hydrolyzing by enzyme for 2.0 h; after the enzymatic hydrolysis, the pH value being adjusted to 9.0 with lye, and standing for 30 min;
(5) filtering: filtering enzymatic hydrolyzed solution in step (4) by using a 40 mesh sieve, and collecting materials on the sieve;
(6) drying: freeze-drying the materials on the sieve, and obtaining a dried product S4 after drying, with a total of 86.2 g;
(7) pulverizing: pulverizing the dried product, sieving through a 100-mesh sieve, and taking the materials under the sieve.

“Ministry of Health Medicine Standard Biochemical Medicines” (1st volume hexose amino acid method for detecting glycosaminoglycan, 1989) is adopted, glycosaminoglycan content is 24.8% (in chondroitin sulfate, coefficient is calculated as 2.82). The method of GB 5009.5 is adopted, the total protein content is 75.1% (the protein conversion coefficient is calculated as 5.79). The method of GB/T 9695.23 is adopted, the hydroxyproline content is 5.9%. The ELISA kit is adopted for detecting, the non-denatured type II collagen content is 23.2%.

### Water Absorption Multiple Detection:

The above-mentioned materials 20 g under the sieve are taken by weighing in the measuring cylinder with scale, purified water is added to sample all wet and be gelatinous. Product volume is rapidly expanded to 256 mL from 40 mL, and volume expansion is 6.4 times. Quantitative filter paper is used to remove free water. After wetting, the mass of the product is 278.2 g and the water absorption is 258.2 g, which is 12.9 times the weight of the product itself.

### Suspension Ability Test:

The above-mentioned materials 20 g under the sieve are taken, mixed with 5% (w/v) liquid with purified water, left standstill 2 h, without layering.

### Example 5

(1) selecting raw materials, where the raw materials to be selected are fresh chicken breast cartilage; cutting 1.0 kg of cartilage within 3 cm from the tip after removing fat and muscle manually;
(2) cleaning: cleaning by using clean water with a temperature not higher than 37° C. for 1 to 3 times, with 10 minutes each time; then draining;
(3) pulverizing: pulverizing the cartilage into cartilage particles below 2 mm by using a pulverizer, after draining the cartilage cleaned in step (2);
(4) hydrolyzing by enzyme: adding 1 L of clean water to the cartilage particles obtained in step (3), making a slurry below 25° C.; placing the slurry into the reaction tank, with the temperature controlled at 35° C.; using acid solution or alkaline solution to adjust a pH value of feed solution between 3.0 and 4.0; adding pepsin-loaded food-grade silica including 20 g of pepsin and 500 g of food-grade silica, stirring and hydrolyzing by enzyme for 2.0 h; after the enzymatic hydrolysis, the pH value being adjusted to 9.0 with lye, and standing for 30 min;
(5) filtering: filtering enzymatic hydrolyzed solution in step (4) by using a 40 mesh sieve, and collecting materials on the sieve;
(6) drying: freeze-drying the materials on the sieve, and obtaining a dried product S5 after drying, with a total of 86.2 g;
(7) pulverizing: pulverizing the dried product, sieving through a 100-mesh sieve, and taking the materials under the sieve.

“Ministry of Health Medicine Standard Biochemical Medicines” (1st volume hexose amino acid method for detecting glycosaminoglycan, 1989) is adopted, glycosaminoglycan content is 25.0% (in chondroitin sulfate, coefficient is calculated as 2.82). The method of GB 5009.5 is adopted, the total protein content is 75.1% (the protein conversion coefficient is calculated as 5.79). The method of GB/T 9695.23 is adopted, the hydroxyproline content is 5.8%. The ELISA kit is adopted for detecting, the non-denatured type II collagen content is 23.5%.

### Water Absorption Multiple Detection:

The above-mentioned materials 20 g under the sieve are taken by weighing in the measuring cylinder with scale, purified water is added to sample all wet and be gelatinous. Product volume is rapidly expanded to 260 mL from 40 mL, and volume expansion is 6.5 times. Quantitative filter paper is used to remove free water. After wetting, the mass of the product is 282 g and the water absorption is 262 g, which is 13.1 times the weight of the product itself.

### Suspension Ability Test:

The above-mentioned materials 20 g under the sieve are taken, mixed with 5% (w/v) liquid with purified water, left standstill 2 h, without layering.

### Comparative Example 1 (Cmp. Ex. 1)

(1) selecting raw materials, where the raw materials to be selected are fresh chicken breast cartilage; cutting 1.0 kg of cartilage 3 cm away from the tip after removing fat and muscle manually;
(2) cleaning: cleaning by using clean water with a temperature not higher than 37° C. for 1 to 3 times, with 10 minutes each time; then draining;
(3) pulverizing: pulverizing the cartilage into cartilage particles below 2 mm by using a pulverizer, after draining the cartilage cleaned in step (2);
(4) hydrolyzing by enzyme: placing the cartilage particles obtained in step (3) into the reaction tank, adding 1 L of clean water, with the temperature controlled at 35° C.; using acid solution or alkaline solution to adjust a pH value of feed solution between 3.0 and 4.0; adding 20 g of pepsin, stirring and hydrolyzing by enzyme for 2.0 h; after the enzymatic hydrolysis, the pH value being adjusted to 9.0 with lye, and standing for 30 min;
(5) filtering: filtering enzymatic hydrolyzed solution in step (4) by using a 40 mesh sieve, and collecting materials on the sieve;
(6) drying: freeze-drying the materials on the sieve, and obtaining a dried product D1 after drying, with a total of 90.1 g;
(7) pulverizing: pulverizing the dried product, sieving through a 100-mesh sieve, and taking the materials under the sieve.

“Ministry of Health Medicine Standard Biochemical Medicines” (1st volume hexose amino acid method for detecting glycosaminoglycan, 1989) is adopted, glycosaminoglycan content is 33.1% (in chondroitin sulfate, coefficient is calculated as 2.82). The method of GB 5009.5 is adopted, the total protein content is 64.2% (the protein conversion coefficient is calculated as 5.79). The method of GB/T 9695.23 is adopted, the hydroxyproline content is 4.1%. The ELISA kit is adopted for detecting, the non-denatured type II collagen content is 6.71%.

### Water Absorption Multiple Detection:

The above-mentioned materials 20 g under the sieve are taken by weighing in the measuring cylinder with scale, purified water is added to sample all wet and be gelatinous. Product volume is rapidly expanded to 156 mL from 38 mL, and volume expansion is 4.1 times. Quantitative filter paper is used to remove free water. After wetting, the mass of the product is 208.1 g and the water absorption is 188.1 g, which is 9.4 times the weight of the product itself.

### Suspension Ability Test:

The above-mentioned materials 20 g under the sieve are taken, mixed with 5% (w/v) liquid with purified water, left standstill 0.5 h, and suspension begins to layer, leave standstill 1.25 h, and suspension is layered with a ratio of 1:1.

### Comparative Example 2

(1) selecting raw materials; selecting 1.0 kg of fresh chicken breast cartilage after removing fat and muscle manually;
(2) cleaning: cleaning by using clean water with a temperature not higher than 37° C. for 1 to 3 times, with 10 minutes each time; then draining;
(3) pulverizing: pulverizing the cartilage into cartilage particles below 2 mm by using a pulverizer, after draining the cartilage cleaned in step (2);
(4) hydrolyzing by enzyme: placing the cartilage particles obtained in step (3) into the reaction tank, adding 1 L of clean water, with the temperature controlled at 35° C.; using acid solution or alkaline solution to adjust a pH value of feed solution between 3.0 and 4.0; adding 20 g of pepsin, stirring and hydrolyzing by enzyme for 2.0 h; after the enzymatic hydrolysis, the pH value being adjusted to 9.0 with lye, and standing for 30 min;
(5) filtering: filtering enzymatic hydrolyzed solution in step (4) by using a 40 mesh sieve, and collecting materials on the sieve;
(6) drying: freeze-drying the materials on the sieve, and obtaining a dried product D2 after drying, with a total of 93.6 g;
(7) pulverizing: pulverizing the dried product, sieving through a 100-mesh sieve, and taking the materials under the sieve.

“Ministry of Health Medicine Standard Biochemical Medicines” (1st volume hexose amino acid method for detecting glycosaminoglycan, 1989) is adopted, glycosaminoglycan content is 30.4% (in chondroitin sulfate, coefficient is calculated as 2.82). The method of GB 5009.5 is adopted, the total protein content is 66.2% (the protein conversion coefficient is calculated as 5.79). The method of GB/T 9695.23 is adopted, the hydroxyproline content is 4.7%. The ELISA kit is adopted for detecting, the non-denatured type II collagen content is 6.9%.

### Water Absorption Multiple Detection:

The above-mentioned materials 20 g under the sieve are taken by weighing in the measuring cylinder with scale, purified water is added to sample all wet and be gelatinous. Product volume is rapidly expanded to 170 mL from 38 mL, and volume expansion is 4.5 times. Quantitative filter paper is used to remove free water. After wetting, the mass of the product is 212 g and the water absorption is 192.0 g, which is 9.6 times the weight of the product itself.

### Suspension Ability Test:

The above-mentioned materials 20 g under the sieve are taken, mixed with 500 (w/v) liquid with purified water, left standstill 47 m2, and suspension begins to layer, leave standstill 65 m, and suspension is layered with a ratio of 1:1.

For different sampling parts, non-denatured type II collagen is prepared by the methods of example 4, and their corresponding product properties are shown in Table 1.

| | S1 | S2 | S3 | S4 | S5 | D1 | |
|---|---|---|---|---|---|---|---|
| | (1.5 cm | (1.5-3 cm | (3 cm | (3 cm | (3 cm | (3 cm | |
| | away | away | away | away | away | away | D2 |
| | from | from | from | from | from | from | (whole |
| Sample/ Item | tip) | tip) | tip) | tip) | tip) | tip) | entire) |
| Glycosaminoglycan/% | 22.3 | 28.9 | 25.2 | 24.8 | 25.0 | 33.1 | 30.4 |
| Total protein/% | 79.1 | 70.3 | 74.5 | 75.1 | 75.1 | 64.2 | 66.2 |
| Hydroxyproline/% | 6.8 | 5.2 | 5.9 | 5.9 | 5.8 | 4.1 | 4.7 |
| Non-denatured type | 21.88 | 16.77 | 18.81 | 23.2 | 23.5 | 6.71 | 6.90 |
| II collagen/% | | | | | | | |
| Volume expansion | 6.2 | 5.1 | 5.3 | 6.4 | 6.5 | 4.1 | 4.5 |
| Water absorption | 12.8 | 11.6 | 12.3 | 12.9 | 13.1 | 9.4 | 9.6 |

The above are only preferred embodiments of the present disclosure, and are not intended to limit the present disclosure. Any simple modifications, changes and equivalent replacements made to the above embodiments according to the technical essence of the present disclosure still fall within the protection scope of the technical solutions of the present disclosure.

### REFERENCES

[1] Liu Bin. High-density fermentation and expression of recombinant human collagen by genetically engineered Pichia pastoris [D]. Nanjing University of Science and Technology, 2012.
[2] Trentham D E. Autoimmunity to type II collagen an experimental model of arthritis. [J]. Journal of Experimental Medicine, 1977, 146(3):857-868.

1. A method for preparing non-denatured type II collagen, comprising steps successively of:
(1) selecting raw materials, where the raw materials to be selected are fresh chicken breast cartilage; cutting a part within 3 cm from a tip of the chicken breast cartilage for use after removing fat and muscle manually;
(2) cleaning: cleaning a spare cartilage in step (1) by using clean water with a temperature not higher than 37° C.;
(3) pulverizing: pulverizing the cartilage into cartilage particles below 2 mm by using a pulverizer, after draining the cartilage cleaned in step (2);
(4) hydrolyzing by enzyme: adding water to the cartilage particles obtained in step (3), with an amount of water added 0.5 to 3 times a mass of the cartilage particles (m/V); using acid solution or alkaline solution to adjust a pH value of feed solution between 2.0 and 4.0;
adding pepsin by weight of 1.0-5.0% cartilage particles, with an enzymatic hydrolysis temperature not exceed 37° C.; stirring and hydrolyzing by enzyme for 0.5-2.0 h;
(5) filtering: filtering enzymatic hydrolyzed solution in step (4) by using a 20-60 mesh sieve, and collecting materials on the sieve;
(6) drying: vacuum-drying or freeze-drying the materials on the sieve, and obtaining a dried product after drying;
(7) pulverizing: pulverizing the dried product, sieving through a 60-200-mesh sieve, and taking the materials under the sieve to obtain non-denatured type II collagen powder.

2. The method for preparing non-denatured type II collagen according to claim 1, wherein, the raw materials are selected from a part within 1.5 cm from the tip of the chicken breast cartilage for use.

3. The method for preparing non-denatured type II collagen according to claim 1, wherein, after enzymolysis finishes, lye is adopted to adjust pH value to 8.5˜9.5, after standing for 15˜60 min, then the filtering of step (5) is performed.

4. The method for preparing non-denatured type II collagen according to claim 1, wherein, the pepsin is added through a load carrier, and the load carrier is food grade silicon dioxide.

5. The method for preparing non-denatured type II collagen according to claim 4, wherein, an addition ratio of the pepsin and the load carrier is (0.3˜0.6):15.

6. The method for preparing non-denatured type II collagen according to claim 1, wherein, in step (4), after adding water to the cartilage particles, beating is performed first, and then acid solution or alkaline solution is adopted to adjust the pH value of feed solution between 2.0 and 4.0.

7. The method for preparing non-denatured type II collagen according to claim 6, wherein, a beating temperature is controlled below 25° C.

8. The method for preparing non-denatured type II collagen according to claim 1, wherein cleaning times in step (2) are 1 to 3 times, with 10 to 15 minutes each time.

9. The method for preparing non-denatured type II collagen according to claim 1, wherein, the enzymatic hydrolysis temperature is 30° C.˜32° C.

10. A non-denatured type II collagen obtained by the method for preparing non-denatured type II collagen according to 1.
